(19) United States
(12) Patent Application Publication
(10) Pub. No.: US 2023/0394193 A1
(43) Pub. Date: Dec. 7, 2023

(54) System And Method For Predicting Physical Properties Of Multilayer Material

(71) Applicant: LG Chem, Ltd., Seoul (KR)

(72) Inventors: Sung Nam Moon, Daejeon (KR); Hyun Tae Kim, Daejeon (KR); Sang Jin Jeon, Daejeon (KR); Jin Mi Jung, Daejeon (KR); Jin Uk Choi, Daejeon (KR); Dam Hyeok Im, Daejeon (KR)

(73) Assignee: LG Chem, Ltd., Seoul (KR)

(21) Appl. No.: 18/032,280

(22) PCT Filed: Aug. 22, 2022
(86) PCT No.: PCT/KR2022/012499

§ 371 Date: Apr. 17, 2023

(30) Foreign Application Priority Data
Aug. 27, 2021 (KR) 10-2021-0113915
Oct. 19, 2021 (KR) 10-2021-0138918
Jul. 13, 2022 (KR) 10-2022-0086165

(51) Int. Cl. G06F 30/20

(52) U.S. Cl. CPC G06F 30/20; G06F 2111/10

(57) ABSTRACT

A system and method for predicting the physical properties of a multilayer material are provided. The system and method can predict physical properties such as a coefficient of thermal expansion and coefficient of water expansion of the multilayer material, and a warpage of the multilayer material when developing the multilayer material.

### CROSS-REFERENCE TO RELATED APPLICATIONS

This application is a national phase entry under 35 U.S.C. § 371 of International Application No. PCT/KR2022/012499, filed on Aug. 22, 2022, which claims priority to and the benefit of Korean Patent Application No. 10-2021-0113915, filed on Aug. 27, 2021, Korean Patent Application No. 10-2021-0138918, filed on Oct. 19, 2021, and Korean Patent Application No. 10-2022-0086165, filed on Jul. 13, 2022, the disclosures of which are incorporated herein by reference in their entireties.

### TECHNICAL FIELD

The present invention relates to a system and method for predicting the physical properties of a multilayer material, and more particularly, to a system and method for predicting the physical properties of a multilayer material when developing the multilayer material.

### BACKGROUND ART

A polymer film refers to a non-fibrous flat plastic molded article, which is light, has a good barrier property, high transparency and is relatively inexpensive, so it is used in many fields such as packaging materials, household goods, electronic devices, automobiles, and aircraft.

Synthetic polymers such as polyethylene (PE), polypropylene (PP), polyvinyl chloride (PVC), and polyethylene terephthalate (PET) are processed into a polymer film and widely used in Korea and abroad, and currently, many synthetic polymers are used as materials for polymer films either alone or by blending.

A multilayer film is a composite film in which different types of films are laminated for the purpose of multi-functionality of the film, and various types of multilayer films formed of, for example, the combination of a film having the excellent mechanical property of polyethylene (PE) and the printing aesthetics of cellophane, and nylon and a vinyl alcohol-ethylene copolymer are used as packaging materials.

In the case of developing such a multilayer film as a material, it is necessary to predict a physical property such as an expansion coefficient of an entire laminate. The expansion coefficient is a value indicating the rate of increase in length or volume of a material when the same is heated or absorbs water. The former is a coefficient of thermal expansion, and the latter is a coefficient of water expansion.

Since multilayer materials show anisotropy in a machine direction (MD) and a transverse direction (TD) in a manufacturing process, for the design of a robust material, it is necessary to predict not only the homogenized stiffness of the entire laminate of a multilayer material, but also warpage that is undesirably generated due to the asymmetric structure caused by thermal and water expansions.

In the process of developing a multilayer material, the homogenized coefficients of thermal expansion and water expansion of the entire laminate are directly related to the deformation of the final product. Therefore, in order to design a robust material, it is necessary to predict warpage as well as thermal expansion and

water expansion considering environmental factors such as a temperature change and a humidity change.

### DISCLOSURE

### Technical Problem

To solve technical problems of the present invention, the present invention is directed to providing a system and method for predicting the physical properties of a multilayer material, which can predict physical properties such as thermal and coefficients of water expansion of an entire laminate when a multilayer material is developed, and predict warpage.

### Technical Solution

In one embodiment for accomplishing the above-described purpose, a system for predicting the physical properties of a multilayer material having n laminated films (n is an integer of 2 or more) according to the present invention includes:
an input unit to which input values including any one or more of an elastic modulus (E^{k}) of each layer (k), a Poisson's ratio (ν^{k}) of each layer (k), a shear modulus (G^{k}) of each layer (k), a thickness (Z^{k}) of each layer (k), and a lamination angle (θ^{k}) of each layer (k), coefficients of thermal expansion (α^{k}_{1,2}) or coefficients of water expansion (β^{k}_{1,2}) of each layer (k), a temperature change (ΔT), or a humidity change (ΔC) are input;
a control unit that calculates the physical properties of the multilayer material by applying input values to the input unit;
a display that is connected to the control unit; and
a storage unit that is connected to the control unit.

In addition, the present invention provides a method of predicting the physical properties of a multilayer material. In one embodiment, the method of predicting the physical properties of a multilayer material having n laminated films (n is an integer of 2 or more) according to the present invention includes inputting input values including any one or more of an elastic modulus (E^{k}) of each layer (k), a Poisson's ratio (ν^{k}) of each layer (k), a shear modulus (G^{k}) of each layer (k), a thickness (Z^{k}) of each layer (k), or a lamination angle (θ^{k}) of each layer (k), coefficients of thermal expansion (α^{k}_{1,2}) or coefficients of water expansion (β^{k}_{1,2}) of each layer (k), a temperature change (ΔT), or a humidity change (ΔC); and
calculating one or more output values of a coefficient of thermal expansion (α) of the multilayer material, a coefficient of water expansion (β) of the multilayer material, or a warpage of the multilayer material by applying the input values.

### Advantageous Effects

The present invention can predict physical properties such as a coefficient of thermal expansion and a coefficient of water expansion of an entire laminate when developing a multilayer material, and predict warpage.

### BRIEF DESCRIPTION OF THE DRAWINGS

FIG. 1 is a diagram illustrating a configuration of a system for predicting the physical properties of a multilayer material according to a first embodiment of the present invention.

FIG. 2 is a diagram illustrating a configuration of an input screen of the system for predicting the physical properties of a multilayer material according to the first embodiment of the present invention.

FIG. 3 is a diagram illustrating a configuration of an output screen of the system for predicting the physical properties of a multilayer material according to the first embodiment of the present invention.

FIG. 4 is a flowchart of a method of predicting the physical properties of a multilayer material according to a second embodiment of the present invention.

FIG. 5 is a flowchart of a method of predicting the physical properties of a multilayer material according to a third embodiment of the present invention.

### DETAILED DESCRIPTION OF THE PREFERRED EMBODIMENTS

In one embodiment for achieving the above-described purposes, a system for predicting the physical properties of a multilayer material having n laminated films (n is an integer of 2 or more) includes an input unit to which input values including any one or more of an elastic modulus (E^{k}) of each layer (k), a Poisson's ratio (ν^{k}) of each layer (k), a shear modulus (G^{k}) of each layer (k), a thickness (Z^{k}) of each layer (k), or a lamination angle (θ^{k}) of each layer (k), coefficients of thermal expansion (α^{k}_{1,2}) or coefficients of water expansion (β^{k}_{1,2}) of each layer (k), a temperature change (ΔT), or a humidity change (ΔC) are input;
a control unit that calculates the physical properties of the multilayer material by applying input values to the input unit;
a display that is connected to the control unit; and
a storage unit that is connected to the control unit.

In addition, the control unit processes the values input to the input unit to calculate any one or more of a coefficient of thermal expansion (α) of the multilayer material, a coefficient of water expansion (β) of the multilayer material, or a warpage of the multilayer material.

In an exemplary embodiment, the values input to the input unit include any one or more of elastic moduli (E^{k}_{1,2}) in the machine direction (1) or the transverse direction (2) of each layer (k),
Poisson's ratios (ν^{k}_{1,2}) in the machine direction (1) or the transverse direction (2) of each layer (k),
shear moduli (G^{K}_{1,2}) in the machine direction (1) or the transverse direction (2) of each layer (k),
an angle (θ^{k}) in the machine direction (1) of each layer with respect to the x direction of the multilayer material, wherein the x direction means an arbitrarily set direction in a plane of the multilayer material, or
a thickness (Z^{k}) of each layer (k); or
any one or more of coefficients of thermal expansion (α^{k}_{1,2}), coefficients of water expansion (β^{k}_{1,2}), a temperature change (ΔT), or a humidity change (ΔC) of each layer (k).

In an exemplary embodiment, to the input unit, elastic moduli (E^{k}_{1,2}) in the machine direction (MD, 1) and the transverse direction (TD, 2) of each layer (k), Poisson's ratios (ν^{k}_{1,2}) in the machine direction (1) and the transverse direction (2) of each layer (k), shear moduli (G^{K}_{1,2}) in the machine direction (1) and the transverse direction (2) of each layer (k), an angle (θ^{k}) in the machine direction (1) of each layer with respect to the x direction of the multilayer material, wherein the x direction means an arbitrarily set direction in a plane of the multilayer material, a thickness (Z^{k}) of each layer (k), coefficients of thermal expansion (α^{k}_{1,2}) and coefficients of water expansion (β^{k}_{1,2}) of each layer (k), a temperature change (ΔT), and a humidity change (ΔC) are input.

In another exemplary embodiment, the control unit calculates stiffness matrices ([Q]^{k}_{1,2}) in the machine direction (1) and the transverse direction (2) of each layer (k) using the elastic moduli (E^{k}_{1,2}), Poisson's ratios (ν^{k}_{1,2}), and shear moduli (G^{k}_{1,2}),
sets inverse matrices ([S]^{k}_{1,2}) for the stiffness matrices ([Q]^{k}_{1,2}) in the machine direction (1) and the transverse direction (2) of each layer (k),
resets stiffness matrices ([Q]^{k}_{x,y}) of each layer (k) by reflecting a lamination angle (θ^{k}) of the multilayer material in the stiffness matrices ([Q]^{k}_{1,2}),
calculates stiffness matrices ([A]_{x,y}, [B]_{x,y}, [D]_{x,y}) of the multilayer material using the values of the reset stiffness matrices by receiving the thickness information of each layer (k),
sets compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material,
calculates free lamina hydrothermal strains (e^{k}_{1,2}) generated by water expansion of each layer (k) in the major direction of each layer (k) using the coefficients of thermal expansion (α^{k}_{1,2}), coefficients of water expansion (β^{k}_{1,2}), temperature change (ΔT), and humidity change (ΔC) of each layer (k),
calculates hygrothermal strain transformations (e^{k}_{x,y,s}) of each layer (k) by reflecting a lamination angle (θ^{k}) of the multilayer material in the free lamina hydrothermal strains (e^{k}_{1,2}),
calculates hygrothermal forces (N^{HT}_{x,y,s}) and hygrothermal moments (M^{HT}_{x,y,s}), generated in the multilayer material, based on the hygrothermal strain transformations (e^{k}_{x,y,s}) of the multilayer material, the stiffness matrices ([Q]^{k}_{x,y}) of the multilayer material, which is the entire laminate, and the thickness (Z^{k}) of each layer (k),
forms total forces (/N) and total moments (/M) by adding external forces (N,M) to the hygrothermal forces (N^{HT}_{x,y,s}) and the hygrothermal moments (M^{HT}_{x,y,s}), and
calculates a coefficient of thermal expansion (α) and coefficient of water expansion (β) of the multilayer material using the total forces (/N) and the total moments (/M), and the compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material.

In still another embodiment, the control unit calculates strains (∈⁰_{x,y}) and curvatures (k_{x,y,s}) of a middle plane using the total forces (/N) and the total moments (/M), and the compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material, and
calculates a warpage of the multilayer material by utilizing the curvatures (k_{x,y,s}) of the middle plane, and sample size (x,y) information.

In yet another embodiment, the control unit also calculates elastic moduli (E_{x,y}), shear moduli (G_{x,y}), and Poisson's ratios (ν_{x,y}) of the multilayer material using the total thickness (h) of the multilayer material and the values of the compliance matrices ([a]_{x,y}, [b]_{x,y}, [c]_{x,y}, [d]_{x,y}).

In one exemplary embodiment, to the input unit, elastic moduli (E^{k}_{1,2}) in the machine direction (MD, 1) and the transverse direction (TD, 2) of each layer (k), Poisson's ratios (ν^{k}_{1,2}) in the machine direction (1) and the transverse direction (2) of each layer (k), shear moduli (G^{K}_{1,2}) in the machine direction (1) and the transverse direction (2) of each layer (k), an angle (θ^{k}) in the machine direction (1) of each layer with respect to the x direction of the multilayer material, wherein the x direction means an arbitrarily set direction in a plane of the multilayer material, a thickness (Z^{k}) of each layer (k), coefficients of thermal expansion (α^{k}_{1,2}) and coefficients of water expansion (β^{k}_{1,2}) of each layer (k), a temperature change (ΔT), and a humidity change (ΔC) are input.

In yet another exemplary embodiment, the control unit calculates stiffness matrices ([Q]^{k}_{1,2}) in the machine direction (1) and the transverse direction (2) of each layer (k) using the elastic moduli (E^{k}_{1,2}), Poisson's ratios (ν^{k}_{1,2}), and shear moduli (G^{k}_{1,2}),
sets inverse matrices ([S]^{k}_{1,2}) for the stiffness matrices ([Q]^{k}_{1,2}) in the machine direction (1) and the transverse direction (2) of each layer (k),
resets stiffness matrices ([Q]^{k}_{x,y}) of each layer (k) by reflecting a lamination angle (θ^{k}) of the multilayer material in the stiffness matrices ([Q]^{k}_{1,2}),
calculates stiffness matrices ([A]_{x,y}, [B]_{x,y}, [D]_{x,y}) of the multilayer material using the values of the reset stiffness matrices by receiving the thickness information of each layer (k),
sets compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material,
calculates elastic moduli (E_{x,y}), shear moduli (G_{x,y}), and Poisson's ratios (ν_{x,y}) of the multilayer material using the total thickness (h) of the multilayer material and the values of the compliance matrices ([a]_{x,y}, [b]_{x,y}, [c]_{x,y}, [d]_{x,y}),
calculates free lamina hydrothermal strains (e^{k}_{1,2}) generated by water expansion of each layer (k) in the major direction of each layer (k) using the coefficients of thermal expansion (α^{k}_{1,2}), coefficients of water expansion (β^{k}_{1,2}), temperature change (ΔT), and humidity change (ΔC) of each layer (k),
calculates hygrothermal strain transformations (e^{k}_{x,y,s}) of each layer (k) by reflecting a lamination angle (θ^{k}) of each layer (k) in the free lamina hydrothermal strains,
calculates hygrothermal forces (N^{HT}_{x,y,s}) and hygrothermal moments (M^{HT}_{x,y,s}) generated in the multilayer material based on the hygrothermal strain transformations (e^{k}_{x,y,s}) of each layer (k), the stiffness matrices ([Q]^{k}_{x,y}) of each layer (k), and the thickness (Z^{k}) of each layer (k),
forms total forces (/N) and total moments (/M) by adding external forces (N,M) to the hygrothermal forces (N^{HT}_{x,y,s}) and the hygrothermal moments (M^{HT}_{x,y,s}),
calculates a coefficient of thermal expansion (α) and coefficient of water expansion (β) of the multilayer material using the total forces (/N) and the total moments (/M), and the compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material,
calculates strains (∈⁰_{x,y}) and curvatures (k_{x,y,s}) of a middle plane using the total forces (/N) and the total moments (/M), and the compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material, and
calculates a warpage of the multilayer material by utilizing the curvature (k_{x,y,s}) of the middle plane, and sample size (x,y) information.

In addition, the present invention provides a method of predicting the physical properties of a multilayer material. In one embodiment, the method of predicting the physical properties of a multilayer material having n laminated films (n is an integer of 2 or more), includes
inputting input values including any one or more of an elastic modulus (E^{k}) of each layer (k), a Poisson's ratio (ν^{k}) of each layer (k), a shear modulus (G^{k}) of each layer (k), a thickness (Z^{k}) of each layer (k), or a lamination angle (θ^{k}) of each layer (k), coefficients of thermal expansion (α^{k}_{1,2}) or coefficients of water expansion (β^{k}_{1,2}) of each layer (k), a temperature change (ΔT), or a humidity change (ΔC); and
calculating any one or more output values of a coefficient of thermal expansion (α) of the multilayer material, a coefficient of water expansion (β) of the multilayer material, or a warpage of the multilayer material by applying the input values.

In one embodiment, in the inputting of input values, the input values include any one or more of elastic moduli (E^{k}_{1,2}) in the machine direction (1) or transverse direction (2) of each layer (k),
Poisson's ratios (ν^{k}_{1,2}) in the machine direction (1) or transverse direction (2) of each layer (k),
shear moduli (G^{K}_{1,2}) in the machine direction (1) or transverse direction (2) of each layer (k),
an angle (θ^{k}) in the machine direction (1) of each layer with respect to the x direction of the multilayer material, wherein the x direction means an arbitrarily set direction in a plane of the multilayer material, or
a thickness (Z^{k}) of each layer (k); or
any one or more of coefficients of thermal expansion (α^{k}_{1,2}), coefficients of water expansion (β^{k}_{1,2}), a temperature change (ΔT), or a humidity change (ΔC) of each layer (k).

In one embodiment, in the method of predicting the physical properties of a multilayer material according to the present invention, the inputting of input values includes inputting elastic moduli (E^{k}_{1,2}) in the machine direction (1) and the transverse direction (2) of each layer (k), Poisson's ratios (ν^{k}_{1,2}) in the machine direction (1) or the transverse direction (2) of each layer (k), shear moduli (G^{K}_{1,2}) in the machine direction (1) or the transverse direction (2) of each layer (k), an angle (θ^{k}) in the machine direction (1) of each layer (k) with respect to the x direction of the multilayer material, and a thickness (Z^{k}) of each layer (k) (S11).

In another embodiment, in the method of predicting the physical properties of a multilayer material according to the present invention, the calculating of output values includes calculating stiffness matrices ([Q]^{k}_{1,2}) in the machine direction (1) and the transverse direction (2) of each layer (k) using the elastic moduli (E^{k}_{1,2}), Poisson's ratios (ν^{k}_{1,2}), and shear moduli (G^{k}_{1,2}) (S12);
setting inverse matrices ([S]^{k}_{1,2}) for the stiffness matrices ([Q]^{k}_{1,2}) in the machine direction (1) and the transverse direction (2) of each layer (k) (S13);
resetting stiffness matrices ([Q]^{k}_{x,y}) of the multilayer material by reflecting a lamination angle (θ^{k}) of each layer (k) in the stiffness matrices ([Q]^{k}_{1,2}) (S14);
calculating stiffness matrices ([A]_{x,y}, [B]_{x,y}, [D]_{x,y}) of the multilayer material using the values of the reset stiffness matrices by receiving the thickness information of each layer (k) (S15);
setting compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material (S16);
inputting coefficients of thermal expansion (α^{k}_{1,2}), coefficients of water expansion (β^{k}_{1,2}), a temperature change (ΔT), and a humidity change (ΔC) of each layer (k) (S21);
calculating free lamina hydrothermal strains (e^{k}_{1,2}) generated by water expansion of each layer (k) in the major direction of each layer (k) using the coefficients of thermal expansion (α^{k}_{1,2}), coefficients of water expansion (β^{k}_{1,2}), temperature change (ΔT), and humidity change (ΔC) of each layer (k) (S22);
calculating hygrothermal strain transformations (e^{k}_{x,y,s}) of the multilayer material by reflecting a lamination angle (θ^{k}) of the multilayer material in the free lamina hydrothermal strains (e^{k}_{1,2}) (S23);
calculating hygrothermal forces (N^{HT}_{x,y,s}) and hygrothermal moments (M^{HT}_{x,y,s}), generated in the multilayer material, based on the hygrothermal strain transformations (e^{k}_{x,y,s}) of the multilayer material, the stiffness matrices ([Q]^{k}_{x,y}) of the multilayer material, which is the entire laminate, and the thickness (Z^{k}) of each layer (k) (S24);
forming total forces (/N) and total moments (/M) by adding external forces (N,M) to the hygrothermal forces (N^{HT}_{x,y,s}) and the hygrothermal moments (M^{HT}_{x,y,s}) (S25); and
calculating a coefficient of thermal expansion (α) and coefficient of water expansion (β) of the multilayer material using the total forces (/N) and the total moments (/M), and the compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material (S26).

In still another embodiment, the method of predicting the physical properties of a multilayer material according to the present invention includes calculating strains (∈⁰_{x,y}) and curvatures (k_{x,y,s}) of a middle plane using the total forces (/N) and the total moments (/M), and the compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material (S27); and
calculating a warpage of the multilayer material by utilizing the curvatures (k_{x,y,s}) of the middle plane, and sample size (x,y) information (S28).

In yet another embodiment, the method of predicting the physical properties of a multilayer material according to the present invention further includes, after the setting of the compliance matrices ([a]_{x,y}, [b]_{x,y}, [c]_{x,y}, [d]_{x,y}) (S16), calculating elastic moduli (E_{x,y}), shear moduli (G_{x,y}), and Poisson's ratios (μ_{x,y}) of the multilayer material using the total thickness (h) of the multilayer material and the values of the compliance matrices ([a]_{x,y}, [b]_{x,y}, [c]_{x,y}, [d]_{x,y}) (S17).

In an exemplary embodiment, in the method of predicting the physical properties of a multilayer material according to the present invention, the inputting of input values includes inputting elastic moduli (E^{k}_{1,2}) in the machine direction (1) and the transverse direction (2) of each layer (k), Poisson's ratios (ν^{k}_{1,2}) in the machine direction (1) and the transverse direction (2) of each layer (k), shear moduli (G^{K}_{1,2}) in the machine direction (1) and the transverse direction (2) of each layer (k), an angle (θ^{k}) in the machine direction (1) of each layer (k) with respect to the x direction of the multilayer material, and a thickness (Z^{k}) of each layer (k) (S11).

In another embodiment, in the method of predicting the physical properties of a multilayer material according to the present invention, the calculating of output values includes calculating stiffness matrices ([Q]^{k}_{1,2}) in the machine direction (1) and the transverse direction (2) of each layer (k) using the elastic moduli (E^{k}_{1,2}), Poisson's ratios (ν^{k}_{1,2}), and shear moduli (G^{k}_{1,2}) (S12);
setting inverse matrices ([S]^{k}_{1,2}) for the stiffness matrices ([Q]^{k}_{1,2}) in the machine direction (1) and the transverse direction (2) of each layer (k) (S13);
resetting stiffness matrices ([Q]^{k}_{x,y}) of the multilayer material by reflecting a lamination angle (θ^{k}) of each layer (k) in the stiffness matrices ([Q]^{k}_{1,2}) (S14);
calculating stiffness matrices ([A]_{x,y}, [B]_{x,y}, [D]_{x,y}) of the multilayer material using the values of the reset stiffness matrices by receiving the thickness information of each layer (k) (S15);
setting compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material (S16);
calculating elastic moduli (E_{x,y}), shear moduli (G_{x,y}), and Poisson's ratios (ν_{x,y}) of the multilayer material using the total thickness (h) of the multilayer material and values of the compliance matrices ([a]_{x,y}, [b]_{x,y}, [c]_{x,y}, [d]_{x,y}) (S17);
inputting coefficients of thermal expansion (α^{k}_{1,2}), coefficients of water expansion (β^{k}_{1,2}), a temperature change (ΔT), and a humidity change (ΔC) of each layer (k) (S21);
calculating free lamina hydrothermal strains (e^{k}_{1,2}) generated by water expansion of each layer (k) in the major direction of each layer (k) using the coefficients of thermal expansion (α^{k}_{1,2}), coefficients of water expansion (β^{k}_{1,2}), temperature change (ΔT), and humidity change (ΔC) of each layer (k) (S22);
calculating hygrothermal strain transformations (e^{k}_{x,y,s}) of the multilayer material by reflecting a lamination angle (θ^{k}) of the multilayer material in the free lamina hydrothermal strains (e^{k}_{1,2}) (S23);
calculating hygrothermal forces (N^{HT}_{x,y,s}) and hygrothermal moments (M^{HT}_{x,y,s}), generated in the multilayer material, based on the hygrothermal strain transformations (e^{k}_{x,y,s}) of the multilayer material, the stiffness matrices ([Q]^{k}_{x,y}) of the multilayer material, which is the entire laminate, and the thickness (Z^{k}) of each layer (k) (S24);
forming total forces (/N) and total moments (/M) by adding external forces (N,M) to the hygrothermal forces (N^{HT}_{x,y,s}) and the hygrothermal moments (M^{HT}_{x,y,s}) (S25);
calculating a coefficient of thermal expansion (α) and coefficient of water expansion (β) of the multilayer material using the total forces (/N) and the total moments (/M), and the compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material (S26);
calculating calculates strains (∈⁰_{x,y}) and curvatures (k_{x,y,s}) of a middle plane using the total forces (/N) and the total moments (/M), and the compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material (S27); and
calculating a warpage of the multilayer material by utilizing the curvature (k_{x,y,s}) of the middle plane, and sample size (x,y) information (S28).

Hereinafter, to describe the technical idea of the present invention in detail so as to be easily implemented by those of ordinary skill in the art to which the present invention belongs, preferred embodiments will be described with reference to the accompanying drawings. Other objects, features, and operational advantages of the present invention, including the object, action, and effect, will be more obvious by the description of preferred embodiments.

For reference, the embodiments disclosed herein are only presented by selecting and presenting the most preferred embodiments to help understanding of those of ordinary skill in the art from among various possible embodiments, and the technical spirit of the present invention is not necessarily limited by the presented embodiments. Various changes, additions, and modifications including equivalents or substitutes are possible without departing from the technical spirit of the present invention.

In addition, terms or words used in in the specification and claims are defined based on the principle in that an inventor can be adequately define the concept of a term in order to describe his/her invention in the best way, and should not be construed as being limited only to the ordinary or dictionary meanings, but should be interpreted with meanings and concepts consistent with the technical spirit of the present invention. As an example, singular expressions include plural expressions unless clearly indicated otherwise in the context, expressions regarding the direction are set based on the position expressed on the drawings for convenience of description, and the expression “connect” or “access” includes not only direct connection or access but also connection or access through a different component between two components. In addition, the expression “unit” includes a unit implemented using hardware, a unit implemented using software, a unit implemented using both hardware and software, and one unit may be implemented using one or more pieces of hardware or software, and two or more units may be implemented using one piece of hardware or software.

### First Embodiment

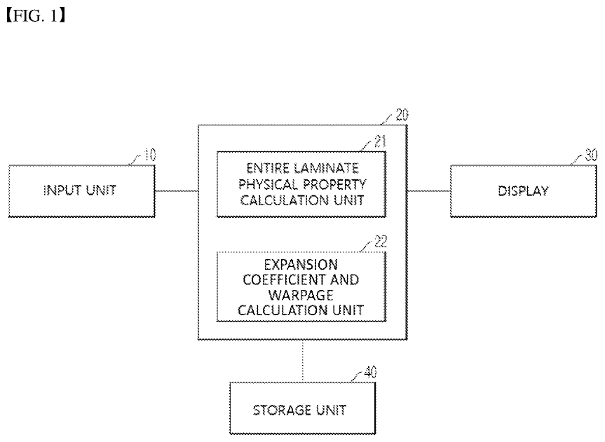

FIG. 1 is a diagram illustrating a configuration of a system for predicting the physical properties of a multilayer material according to a first embodiment of the present invention.

As illustrated in FIG. 1, the configuration of the system for predicting the physical properties of a multilayer material according to the first embodiment of the present invention includes an input unit 10 for inputting elastic moduli (E^{k}_{1,2}) in a machine direction (MD, hereinafter, set as ‘1,’ denoting a major direction) and a transverse direction (TD, hereinafter, set as ‘2’) of each layer (k), Poisson's ratios (ν^{k}_{1,2}) in the machine direction (1) and transverse direction (2) of each layer (k), shear moduli (G^{K}_{1,2}) in the machine direction (1) and transverse direction (2) of each layer (k), an angle (θ^{k}) in the machine direction (1) of each layer with respect to the x direction of the multilayer material, a thickness (Z^{k}) of each layer (k), coefficients of thermal expansion (α^{k}_{1,2}) in the machine direction (1) and transverse direction (2) of each layer (k), coefficients of water expansion (β^{k}_{1,2}) in the machine direction (1) and the transverse direction (2) of each layer (k), a temperature change (ΔT), and a humidity change (ΔC) by a user, a control unit 20, which is connected to the input unit 10 and includes an entire laminate physical property calculation unit 21 and an expansion coefficient and warpage calculation unit 22, a display 30 connected to the control unit 20, and a storage unit 40 connected to the control unit 20.

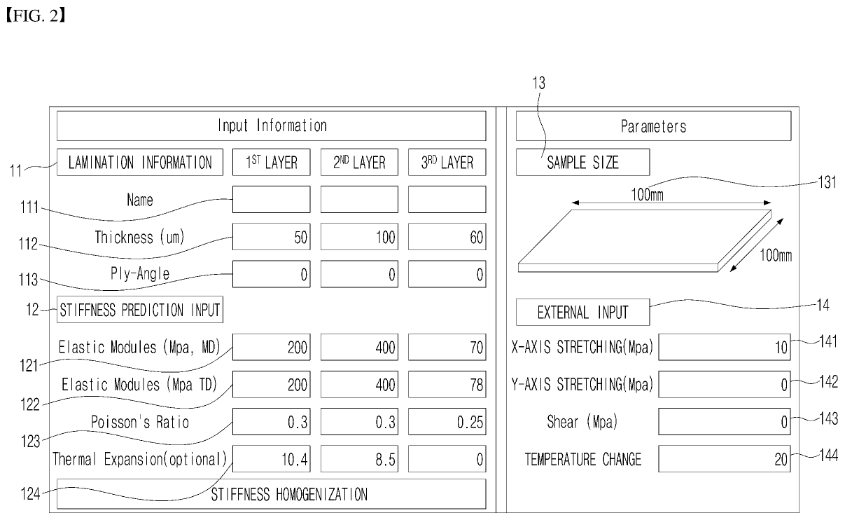

FIG. 2 is a diagram illustrating a configuration of an input screen of the system for predicting the physical properties of a multilayer material according to the first embodiment of the present invention.

As shown in FIG. 2, the configuration of an input screen of the system for predicting the physical properties of a multilayer material according to one embodiment of the present invention is composed of a lamination information input unit 11 in which a name input field 111, a thickness input field 112, and an angle input field 113 are arranged for each layer; a stiffness prediction input unit 12 in which a machine direction (MD) elastic modulus input field 121, a transverse direction (TD) elastic modulus input field 122, a Poisson's ratio input field 123, and a thermal expansion rate input field 124 are arranged for each layer, a sample size input unit 13 in which a sample width and length input field 131 is arranged; and an external force input unit 14 in which an X-axis tensile force input field 141, a Y-axis tensile force input field 142, a shear force input field 143, and a temperature change input field 144 are arranged.

In the case of the input screen of the first embodiment of the present invention, although the multilayer material is set to have three film layers, an input screen having 4 or more film layers may be provided.

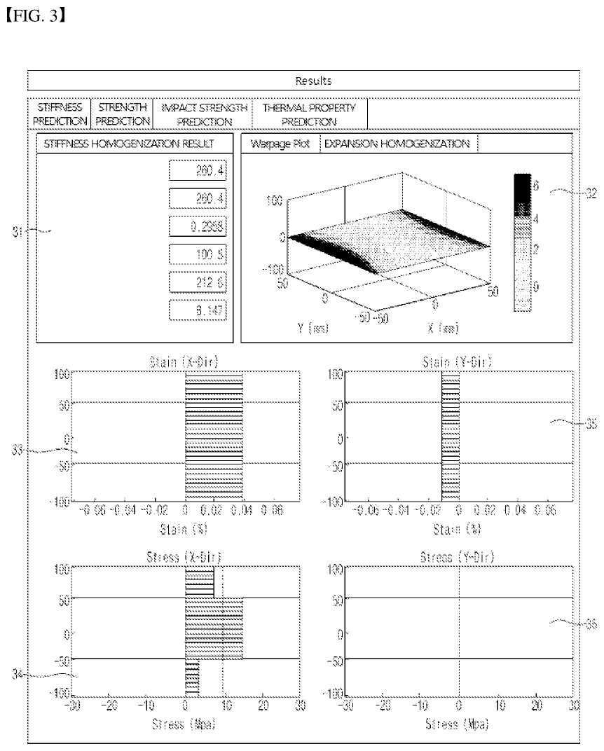

FIG. 3 is a diagram illustrating a configuration of an output screen of the system for predicting the physical properties of a multilayer material according to the first embodiment of the present invention.

As shown in FIG. 3, an output screen of the system for predicting the physical properties of a multilayer material according to the first embodiment of the present invention, as stiffness homogenization results 31, outputs a machine direction (MD) elastic modulus, a transverse (TD) elastic modulus, a Poisson's ratio, a shear modulus, a bulk modulus, and a thermal expansion rate, outputs a warpage plot 32, and outputs a thickness-dependent x-direction strain 33, a thickness-dependent x-direction stress 34, a thickness-dependent y-direction strain 35, and a thickness-dependent y-direction stress 36.

### Second Embodiment

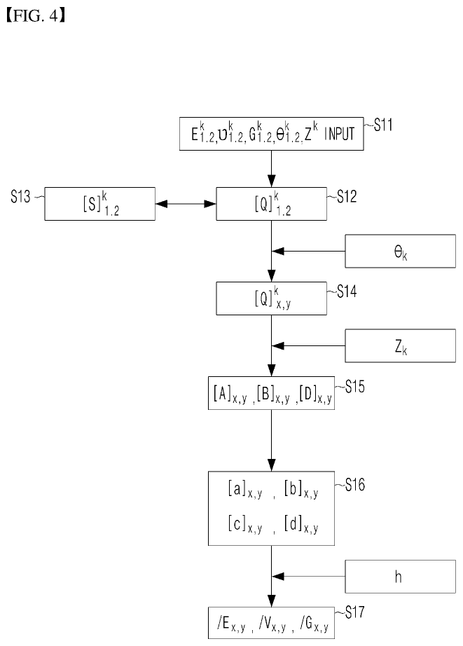

FIG. 4 is a flowchart of a method of predicting the physical properties of a multilayer material according to a second embodiment of the present invention. As shown in FIG. 4, the method of predicting the physical properties of a multilayer material according to the second embodiment of the present invention can be performed as follows.

For the multilayer material in which two or more materials are laminated,
elastic moduli (E^{k}_{1,2}) in the machine direction (1) and the transverse direction (2) of each layer (k), Poisson's ratios (ν^{k}_{1,2}) in the machine direction (1) and the transverse direction (2) of each layer (k), shear moduli (G^{K}_{1,2}) in the machine direction (1) and the transverse direction (2) of each layer (k), an angle (θ^{k}) in the machine direction (1) of each layer (k) with respect to the x direction of the multilayer material, and a thickness (Z^{k}) of each layer (k) are input (S11).

Stiffness matrices ([Q]^{k}_{1,2}) in the machine direction (1) and the transverse direction (2) of each layer (k) are calculated using the elastic moduli (E^{k}_{1,2}), Poisson's ratios (ν^{k}_{1,2}), and shear moduli (G^{k}_{1,2}) as shown in Equation 1 below (S12).

$$[ \begin{matrix}{σ}_{1} \\ {o}_{2} \\ {τ}_{6}\end{matrix} ] = [ \begin{matrix}{Q}_{11} & {Q}_{12} & 0 \\ {Q}_{12} & {Q}_{22} & 0 \\ 0 & 0 & {Q}_{66}\end{matrix} ] [ \begin{matrix}{ε}_{1} \\ {ε}_{2} \\ {γ}_{6}\end{matrix} ] \qquad ( 1 )$$

$${Q}_{11} = \frac{{E}_{1}}{1 - {ν}_{12} {ν}_{21}} {Q}_{22} = \frac{{E}_{2}}{1 - {ν}_{12} {ν}_{21}}$$

$${Q}_{12} = {Q}_{21} = \frac{{ν}_{21} {E}_{1}}{1 - {ν}_{12} {ν}_{21}} = \frac{{ν}_{12} {E}_{2}}{1 - {ν}_{12} {ν}_{21}}$$

$${Q}_{66} = {G}_{12}$$

(For an isotropic material,G=E/2(1+ν))

Inverse matrices ([S]^{k}_{1,2}) for the stiffness matrices ([Q]^{k}_{1,2}) in the machine direction (1) and the transverse direction (2) of each layer (k) that have been calculated as described above are set (S13).

Stiffness matrices ([Q]^{k}_{x,y}) of the multilayer material are reset by reflecting a lamination angle (θ^{k}) of each layer (k) in the obtained stiffness matrices ([Q]^{k}_{1,2}) as shown in Equation (2) below (S14).

$$[ T ] = [ \begin{matrix}{m}^{2} & {n}^{2} & 2 mn \\ {n}^{2} & {m}^{2} & - 2 mn \\ - mn & mn & {m}^{2} - {n}^{2}\end{matrix} ] m = cos θ , n = sin θ \qquad ( 2 )$$

$$[ \begin{matrix}{Q}_{xx} & {Q}_{xy} & 2 {Q}_{xs} \\ {Q}_{xy} & {Q}_{yy} & 2 {Q}_{ys} \\ {Q}_{xs} & {Q}_{ys} & 2 {Q}_{ss}\end{matrix} ] = [ {T}^{- 1} ] [ \begin{matrix}{Q}_{11} & {Q}_{12} & 0 \\ {Q}_{12} & {Q}_{22} & 0 \\ 0 & 0 & 2 {Q}_{66}\end{matrix} ] [ T ]$$

$$[ \begin{matrix}{σ}_{x} \\ {o}_{y} \\ {τ}_{s}\end{matrix} ] = [ \begin{matrix}{Q}_{xx} & {Q}_{xy} & {Q}_{xs} \\ {Q}_{xy} & {Q}_{yy} & {Q}_{ys} \\ {Q}_{xs} & {Q}_{ys} & {Q}_{ss}\end{matrix} ] [ \begin{matrix}{ε}_{x} \\ {ε}_{y} \\ {γ}_{s}\end{matrix} ] \begin{matrix}Stress - strain relation reflecting angle \\ ( dir - x , y )\end{matrix}$$

In addition, the stiffness matrices ([A]_{x,y}, [B]_{x,y}, [D]_{x,y}) of the multilayer material, which is the entire laminate, are calculated using the values of the reset stiffness matrices by receiving the thickness information of each layer (k), as shown in Equation (3) below (S15).

$${A}_{ij} = {\sum }_{k = 1}^{n} {Q}_{ij}^{k} ( {z}_{k} - {z}_{k - 1} ) {B}_{ij} = \frac{1}{2} {\sum }_{k = 1}^{n} {Q}_{ij}^{k} ( {{z}_{k}}^{\hat{}} 2 - {{z}_{k - 1}}^{\hat{}} 2 ) \qquad ( 4 )$$

$${D}_{ij} = \frac{1}{3} {\sum }_{k = 1}^{n} {Q}_{ij}^{k} ( {{z}_{k}}^{\hat{}} 3 - {{z}_{k - 1}}^{\hat{}} 3 )$$

In Equation (3), k indicates each layer, and the multilayer material is formed of a total of n layers, wherein n is an integer of 2 to 10.

The compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material, which is the entire laminate, calculated as described above are set as shown in Equation (4) below (S16).

$${[ \begin{matrix}{A}_{xx} & {A}_{xy} & {A}_{xs} & {B}_{xx} & {B}_{xy} & {B}_{xs} \\ {A}_{yx} & {A}_{yy} & {A}_{ys} & {B}_{yx} & {B}_{yy} & {B}_{ys} \\ {A}_{sx} & {A}_{sy} & {A}_{ss} & {B}_{sx} & {B}_{sy} & {B}_{ss} \\ {B}_{xx} & {B}_{xy} & {B}_{xs} & {D}_{xx} & {D}_{xy} & {D}_{xs} \\ {B}_{yx} & {B}_{yy} & {B}_{ys} & {D}_{yx} & {D}_{yy} & {D}_{ys} \\ {B}_{sx} & {B}_{sy} & {B}_{ss} & {D}_{sx} & {D}_{sy} & {D}_{ss}\end{matrix} ]}^{- 1} = [ \begin{matrix}{a}_{xx} & {a}_{xy} & {a}_{xs} & {b}_{xx} & {b}_{xy} & {b}_{xs} \\ {a}_{yx} & {a}_{yy} & {a}_{ys} & {b}_{yx} & {b}_{yy} & {b}_{ys} \\ {a}_{sx} & {a}_{sy} & {a}_{ss} & {b}_{sx} & {b}_{sy} & {b}_{ss} \\ {c}_{xx} & {c}_{xy} & {c}_{xs} & {d}_{xx} & {d}_{xy} & {d}_{xs} \\ {c}_{yx} & {c}_{yy} & {c}_{ys} & {d}_{yx} & {d}_{yy} & {d}_{ys} \\ {c}_{sx} & {c}_{sy} & {c}_{ss} & {d}_{sx} & {d}_{sy} & {d}_{ss}\end{matrix} ] \qquad ( 4 )$$

If necessary, elastic moduli (/E_{x,y}), shear moduli (/G_{x,y}), and Poisson's ratios (/ν_{x,y}) of the multilayer material, which is the entire laminate, may be calculated using the total thickness (h) of the multilayer material, which is the entire laminate, and the values of the set compliance matrices ([a]_{x,y}, [b]_{x,y}, [c]_{x,y}, [d]_{x,y}) as shown in Equation (5) below.

$$/ {E}_{x} = \frac{1}{{ha}_{xx}} / {E}_{y} = \frac{1}{{ha}_{yy}} / {G}_{xy} = \frac{1}{{ha}_{ss}} \qquad ( 5 )$$

$$/ {ν}_{xy} = - \frac{{a}_{yx}}{{a}_{xx}} / {ν}_{yx} = - \frac{{a}_{xy}}{{a}_{yy}}$$

In the inputting of the elastic moduli (E^{k}_{1,2}), Poisson's ratios (ν^{k}_{1,2}), and shear moduli (G^{K}_{1,2}) of each layer (k) (S11), the shear moduli (G^{k}_{1,2}) are calculated by a control unit 20 using the following relation between the elastic modulus (E), the shear modulus (G), and the Poisson's ratio (ν) of an isotropic material.

$$G = \frac{E}{2 ( 1 + ν )}$$

### Third Embodiment

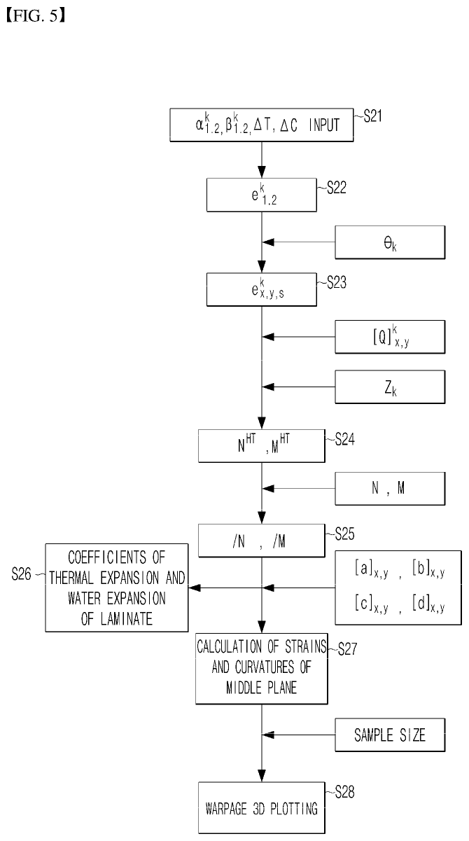

FIG. 5 is a flowchart of a method of predicting the physical properties of a multilayer material according to a third embodiment of the present invention. As shown in FIG. 5, the method of predicting the physical properties of a multilayer material according to a third embodiment of the present invention is as follows.

For the multilayer material in which two or more films are laminated, inputting coefficients of thermal expansion (α^{k}_{1,2}), coefficients of water expansion (β^{k}_{1,2}), a temperature change (ΔT), and a humidity change (ΔC) of each layer (k) (S21) is performed.

Free lamina hydrothermal strains (e^{k}_{1,2}) generated by water expansion of each layer (k) in the major direction of each layer (k) are calculated using the input coefficients of thermal expansion (α^{k}_{1,2}), coefficients of water expansion (β^{k}_{1,2}), temperature change (ΔT), and humidity change (ΔC) of each layer (k), as shown in Equation (6) below (S22).

e₁^{k}=α₁^{k}ΔT+β₁^{k}ΔC e₂^{k}=α₁^{k}ΔT+β₁^{k}ΔC (6)

Hygrothermal strain transformations (e^{k}_{x,y,s}) of the multilayer material, which is the entire laminate, are calculated by reflecting a lamination angle (θ^{k}) of each layer (k) in the calculated free lamina hydrothermal strains, as shown in Equation (7) (S23).

eₓ^{k}=e₁^{k}m²+e₂^{k}n² m=cos θ,n=sin θ

e_{y}^{k}=e₁^{k}n²+e₂^{k}m²

eₛ^{k}=2(e₁^{k}+e₂^{k})mn (7)

Based on the hydrothermal strain transformations (e^{k}_{x,y,s}) of the multilayer material, which is the entire laminate, the stiffness matrices ([Q]^{k}_{x,y}) of the multilayer material, which is the entire laminate, calculated in the second embodiment, and the thickness (Z^{k}) of each layer (k), hygrothermal forces (N^{HT}_{x,y,s}) and hygrothermal moments (M^{HT}_{x,y,s}), generated in the multilayer material, which is the entire laminate, are calculated as shown in Equation (8) below (S24).

$$[ \begin{matrix}\begin{matrix}{N}_{x}^{HT} \\ {N}_{y}^{HT}\end{matrix} \\ {N}_{s}^{HT}\end{matrix} ] = {\sum }_{k = 1}^{n} {{[ \begin{matrix}{Q}_{xx} & {Q}_{xy} & {Q}_{xs} \\ {Q}_{xy} & {Q}_{yy} & {Q}_{ys} \\ {Q}_{xs} & {Q}_{ys} & {Q}_{ss}\end{matrix} ]}_{k} [ \begin{matrix}\begin{matrix}{e}_{x} \\ {e}_{y}\end{matrix} \\ {e}_{s}\end{matrix} ]}_{k} {t}_{k} \qquad ( 8 )$$

$$[ \begin{matrix}\begin{matrix}{M}_{x}^{HT} \\ {M}_{y}^{HT}\end{matrix} \\ {M}_{s}^{HT}\end{matrix} ] = {\sum }_{k = 1}^{n} {{[ \begin{matrix}{Q}_{xx} & {Q}_{xy} & {Q}_{xs} \\ {Q}_{xy} & {Q}_{yy} & {Q}_{ys} \\ {Q}_{xs} & {Q}_{ys} & {Q}_{ss}\end{matrix} ]}_{k} [ \begin{matrix}\begin{matrix}{e}_{x} \\ {e}_{y}\end{matrix} \\ {e}_{s}\end{matrix} ]}_{k} {z}_{k} {t}_{k}$$

Total forces (/N) and total moments (/M) are formed by adding external forces (N,M), which are mechanical loads, to the hygrothermal forces (N^{HT}_{x,y,s}) and hygrothermal moments (M^{HT}_{x,y,s}) calculated above as shown in Equation (9) below (S25).

/N=N+N^{HT}/M=M+M^{HT} (9)

Using the total forces (/N) and the total moments (/M), and the compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material, which is the entire laminate, calculated in the second embodiment, a coefficient of thermal expansion (α) of the multilayer material, which is the entire laminate, is obtained as shown in Equation (10) below.

$$[ \begin{matrix}\begin{matrix}/ {α}_{x} \\ / {α}_{s}\end{matrix} \\ / {α}_{s}\end{matrix} ] = [ \begin{matrix}{α}_{xx} & {α}_{xy} & {α}_{xs} \\ {α}_{yx} & {α}_{yy} & {α}_{ys} \\ {α}_{sx} & {α}_{sy} & {α}_{ss}\end{matrix} ] [ \begin{matrix}\begin{matrix}{N}_{x}^{T} \\ {N}_{y}^{T}\end{matrix} \\ {N}_{s}^{T}\end{matrix} ] + [ \begin{matrix}{b}_{xx} & {b}_{xy} & {b}_{xs} \\ {b}_{yx} & {b}_{yy} & {b}_{ys} \\ {b}_{sx} & {b}_{sy} & {b}_{ss}\end{matrix} ] [ \begin{matrix}\begin{matrix}{M}_{x}^{T} \\ {M}_{y}^{T}\end{matrix} \\ {M}_{s}^{T}\end{matrix} ] \qquad ( 10 )$$

(Here, provided that [N]=0, [M]=0, ΔT=1)

A coefficient of water expansion (β) of the multilayer material, which is the entire laminate, is calculated as shown in Equation (11) below (S26).

$$w = - \frac{1}{2} ( {k}_{x} {x}^{2} + {k}_{y} {y}^{2} + {k}_{s} x y ) \qquad ( 11 )$$

(Here, provided that [N]=0, [M]=0, ΔC=1)

Using the total forces (/N) and the total moments (/M), and the compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material, which is the entire laminate, calculated in the second embodiment, strains (∈_{x,y}) and curvatures (k_{x,y,s}) of a middle plane are calculated as shown in Equation (12) below (S27).

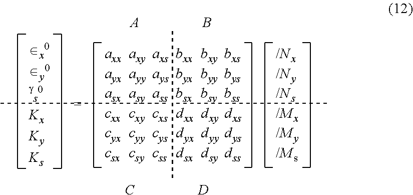

A warpage is calculated as shown in Equation (13) and then 3D plotted by utilizing the curvatures of the middle plane, and the sample size (x,y) information input through the input unit 10 (S28).

$$w = - \frac{1}{2} ( {k}_{x} {x}^{2} + {k}_{y} {y}^{2} + {k}_{s} x y ) \qquad ( 13 )$$

### Fourth Embodiment

The actions of the system and method for predicting the physical properties of a multilayer material according to the embodiments of the present invention, which have been configured as above, are as follows.

To predict the physical properties of the multilayer material, a user inputs elastic moduli (E^{k}_{1,2}) in the machine direction (1) and transverse direction (2) of each layer (k, k=1,2,3), Poisson's ratios (ν^{k}_{1,2}) in the machine direction (1) and transverse direction (2) of each layer (k, k=1,2,3), shear moduli (G^{K}_{1,2}) in the machine direction (1) and transverse direction (2) of each layer (k, k=1,2,3), an angle (θ^{k}) in the machine direction (1) of each layer (k, k=1,2,3) with respect to the x direction of the multilayer material, and a thickness (Z^{k}) of each layer (k) using an input unit 10 while looking at an input screen on a display 30 (S11).

On the input screen provided by the display 30, as shown in FIG. 2, a lamination data input unit 11 in which a name input field 111, a thickness input field 112, and an angle input field 113 are arranged for each layer, a stiffness prediction input unit 12 in which a machine direction (MD) elastic modulus input field 121, a transverse direction (TD) elastic modulus input field 122, a Poisson's ratio input field 123, and a thermal expansion rate input field 124 are arranged for each layer, a sample size input unit 13 in which a sample width and length input field 131 is arranged; and an external force input unit 14 in which an X-axis tensile force input field 141, a Y-axis tensile force input field 142, a shear force input field 143, and a temperature change input field 144 are arranged are provided, information that does not appear on the input screen is automatically calculated by the control unit 20 and stored in a storage unit 40. For example, when the machine direction (MD) elastic modulus (E^{k}₁), transverse direction (TD) elastic modulus (E^{k}₂), and Poisson's ratios (ν^{k}_{1,2}) are input for each layer through the input screen and the input unit 10, a control unit 20 automatically calculates shear moduli (G^{k}_{1,2}) using the relationship between an elastic modulus (E), a shear modulus (G) and a Poisson's ratio (ν) of an isotropic material below, and then stores the same in the storage unit 40.

$$G = \frac{E}{2 ( 1 + ν )}$$

Next, an entire laminate physical property calculation unit 21 of the control unit 20 calculates stiffness matrices ([Q]^{k}_{1,2}) in the machine direction (1) and the transverse direction (2) of each layer (k) using the elastic moduli (E^{k}_{1,2}), Poisson's ratios (ν^{k}_{1,2}), and shear moduli (G^{k}_{1,2}) as shown in Equation (1) below (S12).

$$[ \begin{matrix}{σ}_{1} \\ {o}_{2} \\ {τ}_{6}\end{matrix} ] = [ \begin{matrix}{Q}_{11} & {Q}_{12} & 0 \\ {Q}_{12} & {Q}_{22} & 0 \\ 0 & 0 & {Q}_{66}\end{matrix} ] [ \begin{matrix}{ε}_{1} \\ {ε}_{2} \\ {γ}_{6}\end{matrix} ] \qquad ( 1 )$$

$${Q}_{11} = \frac{{E}_{1}}{1 - {ν}_{12} {ν}_{21}} {Q}_{22} = \frac{{E}_{2}}{1 - {ν}_{12} {ν}_{21}}$$

$${Q}_{12} = {Q}_{21} = \frac{{ν}_{21} {E}_{1}}{1 - {ν}_{12} {ν}_{21}} = \frac{{ν}_{12} {E}_{2}}{1 - {ν}_{12} {ν}_{21}}$$

$${Q}_{66} = {G}_{12}$$

(The relation for an isotropic material G=E/2(1+ν) is used)

Subsequently, the entire laminate physical property calculation unit 21 of the control unit 20 obtains inverse matrices ([S]^{k}_{1,2}) for the calculated stiffness matrices ([Q]^{k}_{1,2}) in the machine direction (1) and the transverse direction (2) of each layer (k), calculated above, and set as compliance matrices (S13). This is for transforming a stiffness matrix, which is a singular matrix, into an invertible matrix in which an inverse matrix is present.

Afterward, the entire laminate physical property calculation unit 21 of the control unit 20 resets stiffness matrices ([Q]^{k}_{x,y}) of each layer (k) by reflecting a lamination angle (θ^{k}) of the multilayer material in the obtained stiffness matrices ([Q]^{k}_{1,2}) of each layer (k), as shown in Equation (2) below (S14).

$$[ T ] = [ \begin{matrix}{m}^{2} & {n}^{2} & 2 mn \\ {n}^{2} & {m}^{2} & - 2 mn \\ - mn & mn & {m}^{2} - {n}^{2}\end{matrix} ] m = cos θ , n = sin θ \qquad ( 2 )$$

$$[ \begin{matrix}{Q}_{xx} & {Q}_{xy} & 2 {Q}_{xs} \\ {Q}_{xy} & {Q}_{yy} & 2 {Q}_{ys} \\ {Q}_{xs} & {Q}_{ys} & 2 {Q}_{ss}\end{matrix} ] = [ {T}^{- 1} ] [ \begin{matrix}{Q}_{11} & {Q}_{12} & 0 \\ {Q}_{12} & {Q}_{22} & 0 \\ 0 & 0 & 2 {Q}_{66}\end{matrix} ] [ T ]$$

$$[ \begin{matrix}{σ}_{x} \\ {o}_{y} \\ {τ}_{s}\end{matrix} ] = [ \begin{matrix}{Q}_{xx} & {Q}_{xy} & {Q}_{xs} \\ {Q}_{xy} & {Q}_{yy} & {Q}_{ys} \\ {Q}_{xs} & {Q}_{ys} & {Q}_{ss}\end{matrix} ] [ \begin{matrix}{ε}_{x} \\ {ε}_{y} \\ {γ}_{s}\end{matrix} ] \begin{matrix}Stress - strain relation reflecting angle \\ ( dir - x , y )\end{matrix}$$

Subsequently, the entire laminate physical property calculation unit 21 of the control unit 20 calculates stiffness matrices ([A]_{x,y}, [B]_{x,y,} [D]_{x,y}) of the multilayer material, which is the entire laminate, using values of the reset stiffness matrices by receiving the thickness information of each layer (k), as shown in Equation (3) below (S15).

$$\\ \begin{matrix}{A}_{ij} = {\sum }_{k = 1}^{n} {Q}_{ij}^{k} ( {z}_{k} - {z}_{k - 1} ) {B}_{ij} = \frac{1}{2} {\sum }_{k = 1}^{n} {Q}_{ij}^{k} ( {{z}_{k}}^{\hat{}} 2 - {{z}_{k - 1}}^{\hat{}} 2 ) & ( 3 )\end{matrix}$$

$${D}_{ij} = \frac{1}{3} {\sum }_{k = 1}^{n} {Q}_{ij}^{k} ( {{z}_{k}}^{\hat{}} 3 - {{z}_{k - 1}}^{\hat{}} 3 )$$

In Equation (3), k indicates each layer, and the multilayer material is formed of a total of n layers, and n is an integer of 2 to 10.

Then, the entire laminate physical property calculation unit 21 of the control unit 20 sets compliance matrices ([a]_{x,y}, [b]_{x,y}, [c]_{x,y}, [d]_{x,y}) for the calculated stiffness matrices ([A]_{x,y}, [B]_{x,y}, [D]_{x,y}) of the multilayer material, which is the entire laminate, as shown in Equation (4) (S16).

$$\\ \begin{matrix}{[ \begin{matrix}{A}_{xx} & {A}_{xy} & {A}_{xs} & {B}_{xx} & {B}_{xy} & {B}_{xs} \\ {A}_{yx} & {A}_{yy} & {A}_{ys} & {B}_{yx} & {B}_{yy} & {B}_{ys} \\ {A}_{sx} & {A}_{sy} & {A}_{ss} & {B}_{sx} & {B}_{sy} & {B}_{ss} \\ {B}_{xx} & {B}_{xy} & {B}_{xs} & {D}_{xx} & {D}_{xy} & {D}_{xs} \\ {B}_{yx} & {B}_{yy} & {B}_{ys} & {D}_{yx} & {D}_{yy} & {D}_{ys} \\ {B}_{sx} & {B}_{sy} & {B}_{ss} & {D}_{sx} & {D}_{sy} & {D}_{ss}\end{matrix} ]}^{- 1} = [ \begin{matrix}{a}_{xx} & {a}_{xy} & {a}_{xs} & {b}_{xx} & {b}_{xy} & {b}_{xs} \\ {a}_{yx} & {a}_{yy} & {a}_{ys} & {b}_{yx} & {b}_{yy} & {b}_{ys} \\ {a}_{sx} & {a}_{sy} & {a}_{ss} & {b}_{sx} & {b}_{sy} & {b}_{ss} \\ {c}_{xx} & {c}_{xy} & {c}_{xs} & {d}_{xx} & {d}_{xy} & {d}_{xs} \\ {c}_{yx} & {c}_{yy} & {c}_{ys} & {d}_{yx} & {d}_{yy} & {d}_{ys} \\ {c}_{sx} & {c}_{sy} & {c}_{ss} & {d}_{sx} & {d}_{sy} & {d}_{ss}\end{matrix} ] & ( 4 )\end{matrix}$$

Subsequently, the entire laminate physical property calculation unit 21 of the control unit 20 obtains a total thickness (h) of the multilayer material, which is the entire laminate, from the thickness information of each layer (k), and calculates elastic moduli (/E_{x,y}), shear moduli (/G_{x,y}), and Poisson's ratios (/ν_{x,y}) of the multilayer material, which is the entire laminate, using values of the compliance matrices ([a]_{x,y}, [b]_{x,y}, [c]_{x,y}, [d]_{x,y}) set above, as shown in Equation (5) below (S17).

$$\\ \begin{matrix}/ {E}_{x} = \frac{1}{{ha}_{xx}} / {E}_{y} = \frac{1}{{ha}_{yy}} / {G}_{xy} = \frac{1}{{ha}_{ss}} / {v}_{xy} = - \frac{{a}_{yx}}{{a}_{xx}} / {v}_{yx} = - \frac{{a}_{xy}}{{a}_{yy}} & ( 5 )\end{matrix}$$

Next, the user inputs coefficients of thermal expansion (α^{k}_{1,2}), coefficients of water expansion (β^{k}_{1,2}), a temperature change (ΔT), and a humidity change (ΔC) of each layer (k) using the input unit 10 while looking at an input screen (not shown) on the display 30 (S21), and then the expansion coefficient and warpage calculation unit 22 of the control unit 20 reads the input values.

Subsequently, the expansion coefficient and warpage calculation unit 22 of the control unit 20 calculates free lamina hydrothermal strains (e^{k}_{1,2}) caused by the water expansion of each layer (k) in the major direction of each layer (k) using the input coefficient of thermal expansion (α^{k}_{1,2}), coefficient of water expansion (β^{k}_{1,2}), temperature change (ΔT), and humidity change (ΔC) of each layer (k) as shown in Equation (6) below (S22).

e₁^{k}=α₁^{k}ΔT+β₁^{k}ΔC e₂^{k}=α₁^{k}ΔT+β₁^{k}ΔC (6)

Next, the expansion coefficient and warpage calculation unit 22 of the control unit 20 calculates hygrothermal strain transformations (e^{k}_{x,y,s}) of the multilayer material, which is the entire laminate, by reflecting a lamination angle of each layer (k) in the calculated free lamina hydrothermal strains as shown in Equation (7) below (S23).

eₓ^{k}=e₁^{k}m²+e₂^{k}n² m=cos θ,n=sin θ

e_{y}^{k}=e₁^{k}n²+e₂^{k}m²

eₛ^{k}=2(e₁^{k}+e₂^{k})mn (7)

Subsequently, the expansion coefficient and warpage calculation unit 22 of the control unit 20 calculates hygrothermal forces (N^{HT}_{x,y,s}) and hygrothermal moments (M^{HT}_{x,y,s}), generated in the multilayer material, which is the entire laminate, based on the hygrothermal strain transformations (e^{k}_{x,y,s}) of the multilayer material, and the stiffness matrices ([Q]^{k}_{x,y}) of the multilayer material, which is the entire laminate, calculated in the second embodiment, and the thickness (Z^{k}) of each layer (k) as shown in Equation (8) below (S24).

$$\\ \begin{matrix}[ \begin{matrix}{N}_{x}^{HT} \\ {N}_{y}^{HT} \\ {N}_{s}^{HT}\end{matrix} ] = {\sum }_{k = 1}^{n} {{[ \begin{matrix}{Q}_{xx} & {Q}_{xy} & {Q}_{xs} \\ {Q}_{xy} & {Q}_{yy} & {Q}_{ys} \\ {Q}_{xs} & {Q}_{ys} & {Q}_{ss}\end{matrix} ]}_{k} [ \begin{matrix}{e}_{x} \\ {e}_{y} \\ {e}_{s}\end{matrix} ]}_{k} {t}_{k} & ( 8 )\end{matrix}$$

$$\\ [ \begin{matrix}{M}_{x}^{HT} \\ {M}_{y}^{HT} \\ {M}_{s}^{HT}\end{matrix} ] = {\sum }_{k = 1}^{n} {{[ \begin{matrix}{Q}_{xx} & {Q}_{xy} & {Q}_{xs} \\ {Q}_{xy} & {Q}_{yy} & {Q}_{ys} \\ {Q}_{xs} & {Q}_{ys} & {Q}_{ss}\end{matrix} ]}_{k} [ \begin{matrix}{e}_{x} \\ {e}_{y} \\ {e}_{s}\end{matrix} ]}_{k} {z}_{k} {t}_{k}$$

Then, the layer strain and stress calculation unit 22 of the control unit 20 forms total forces (/N) and total moments (/M) by adding external forces (N,M), which are mechanical loads, to the calculated hygrothermal forces (N^{HT}_{x,y,s}) and hygrothermal moments (M^{HT}_{x,y,s}) as shown in Equation (9) below (S25).

/N=N+N^{HT}/M=M+M^{HT} (9)

Subsequently, the expansion coefficient and warpage calculation unit 22 of the control unit 20 obtains a coefficient of thermal expansion (/α) of the multilayer material, which is the entire laminate, using the total forces (/N) and the total moments (/M), and the compliance matrices ([a]_{x,y}, [b]_{x,y}, [c]_{x,y}, [d]_{x,y}) for the stiffness matrices ([A]_{x,y}, [B]_{x,y}, [C]_{x,y}) of the multilayer material, which is the entire laminate, calculated in the second embodiment as shown in Equation (10) below, and

$$\\ \begin{matrix}[ \begin{matrix}/ {α}_{x} \\ / {α}_{s} \\ / {α}_{s}\end{matrix} ] = [ \begin{matrix}{α}_{xx} & {α}_{xy} & {α}_{xs} \\ {α}_{yx} & {α}_{yy} & {α}_{ys} \\ {α}_{sx} & {α}_{sy} & {α}_{ss}\end{matrix} ] [ \begin{matrix}{N}_{x}^{T} \\ {N}_{y}^{T} \\ {N}_{s}^{T}\end{matrix} ] + [ \begin{matrix}{b}_{xx} & {b}_{xy} & {b}_{xs} \\ {b}_{yx} & {b}_{yy} & {b}_{ys} \\ {b}_{sx} & {b}_{sy} & {b}_{ss}\end{matrix} ] [ \begin{matrix}{M}_{x}^{T} \\ {M}_{y}^{T} \\ {M}_{s}^{T}\end{matrix} ] & ( 10 )\end{matrix}$$

(Here, provided that [N]=0, [M]=1)
also obtains a coefficient of water expansion (β) of the multilayer material, which is the entire laminate, as shown in Equation (11) below (S26).

$$\\ \begin{matrix}[ \begin{matrix}{\overline{β}}_{x} \\ {\overline{β}}_{s} \\ {\overline{β}}_{s}\end{matrix} ] = [ \begin{matrix}{α}_{xx} & {α}_{xy} & {α}_{xs} \\ {α}_{yx} & {α}_{yy} & {α}_{ys} \\ {α}_{sx} & {α}_{sy} & {α}_{ss}\end{matrix} ] [ \begin{matrix}{N}_{x}^{H} \\ {N}_{y}^{H} \\ {N}_{s}^{H}\end{matrix} ] + [ \begin{matrix}{b}_{xx} & {b}_{xy} & {b}_{xs} \\ {b}_{yx} & {b}_{yy} & {b}_{ys} \\ {b}_{sx} & {b}_{sy} & {b}_{ss}\end{matrix} ] [ \begin{matrix}{M}_{x}^{H} \\ {M}_{y}^{H} \\ {M}_{s}^{H}\end{matrix} ] & ( 11 )\end{matrix}$$

(Here, provided that [N]=0, [M]=0, ΔC=1)

Then, the expansion coefficient and warpage calculation unit 22 of the control unit 20 calculates strains (∈⁰_{x,y}) and curvatures (k_{x,y,s}) of a middle plane using the total forces (/N) and the total moments (/M), and the compliance matrices ([a]_{x,y}, [b]_{x,y}, [c]_{x,y}, [d]_{x,y}) for the stiffness matrices ([A]_{x,y}, [B]_{x,y}, [C]_{x,y}) of the multilayer material, which is the entire laminate, calculated in the second embodiment as shown in Equation 12 below (S27).

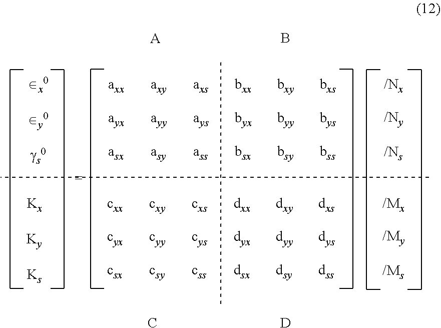

Subsequently, the expansion coefficient and warpage calculation unit 22 of the control unit 20 calculates a warpage (w) by utilizing the curvatures (k_{x,y,s}) of the middle plate, and the sample size (x,y) information input through the input unit 10 as shown in Equation (13) below and then 3D plotted on an output image (S28).

$$\\ \begin{matrix}W = - \frac{1}{2} ( {k}_{x} {x}^{2} + {k}_{y} {y}^{2} + {k}_{s} xy ) & ( 13 )\end{matrix}$$

On the output image provided in the display 30, as shown in FIG. 3, as stiffness homogenization results 31, a machine direction (MD) elastic modulus, a transverse (TD) elastic modulus, a Poisson's ratio, a shear modulus, a bulk modulus, and a thermal expansion rate are outputted, a warpage plot 32 is outputted, and a thickness-dependent x-direction strain 33, a thickness-dependent x-direction stress 34, a thickness-dependent y-direction strain 35, and a thickness-dependent y-direction stress 36 are output.

### Fifth Embodiment

In one embodiment of the present invention, when the values input to the input unit are not immediately obtained, they may be deduced through a conversion process using other physical property values.

In one embodiment, in the process of inputting the elastic modulus (E^{k}) of each layer (k) and the Poisson's ratio (ν^{k}) of each layer (k), these values can be calculated using one or more physical property values of a Lame's first parameter (λ^{k}), a shear modulus (G^{k}) or a bulk elastic modulus (K^{k}). For example, they can be converted into an elastic modulus (E^{k}) and a Poisson's ratio (ν^{k}) using Formulas 1 to 9 below.

When the available combination is (λ^{k}, G^{k}), it is expressed by Formula 1 below.

$$\\ \begin{matrix}{E}^{k} = \frac{{G}^{k} ( 3 {λ}^{k} + 2 {G}^{k} )}{{λ}^{k} + {G}^{k} )} , {v}^{k} = \frac{{λ}^{k}}{2 ( {λ}^{k} + {G}^{k} )} & [ Formula 1 ]\end{matrix}$$

When the available combination is (λ^{k}, E^{k}), it is expressed by Formula 2 below.

$$\\ \begin{matrix}{v}^{k} = \frac{A - ( {E}^{k} + {λ}^{k} )}{4 {λ}^{k}} , {E}^{k} & [ Formula 2 ]\end{matrix}$$

When the available combination is (λ^{k}, ν^{k}), it is expressed by Formula 3 below.

$$\\ \begin{matrix}{E}^{k} = \frac{{λ}^{k} ( 1 + {λ}^{k} ) ( 1 - 2 {λ}^{k} )}{{λ}^{k}} , {v}^{k} & [ Formula 3 ]\end{matrix}$$

When the available combination is (λ^{k}, K^{k}), it is expressed by Formula 4 below.

$$\\ \begin{matrix}{E}^{k} = \frac{9 {K}^{k} ( {K}^{k} - {λ}^{k} )}{3 {K}^{k} - {λ}^{k}} , {v}^{k} = \frac{{λ}^{k}}{3 {K}^{k} - {λ}^{k}} & [ Formula 4 ]\end{matrix}$$

When the available combination is (G^{k}, E^{k}), it is expressed by Formula 5 below.

$$\\ \begin{matrix}{v}^{k} = \frac{E - 2 G}{2 {G}^{k}} , {E}^{k} & [ Formula 5 ]\end{matrix}$$

When the available combination is (G^{k}, ν^{k}), it is expressed by Formula 6 below.

E^{k}=2G^{k}(1+ν^{k}),ν^{k} [Formula 6]

When the available combination is (G^{k}, K^{k}), it is expressed by Formula 7 below.

$${E}^{k} = \frac{9 {K}^{k} {G}^{k}}{3 {K}^{k} + {G}^{k}} , {v}^{k} = \frac{3 {K}^{k} - 2 {G}^{k}}{2 ( 3 {K}^{k} + {G}^{k} )} \qquad [ Formula 7 ]$$

When the available combination is (K^{k}, E^{k}), it is expressed by Formula 8 below.

$${v}^{k} = \frac{3 {K}^{k} - {E}^{k}}{6 {K}^{k}} , {E}^{k} \qquad [ Formula 8 ]$$

When the available combination is (K^{k}, ν^{k}), it is expressed by Formula 9 below.

E^{k}=3K^{k}(1−2ν^{k})ν^{k} [Formula 9]

The Formulas 1 to 9 above are examples, and it is possible to combine two or more formulas as needed.

### DESCRIPTION OF REFERENCE NUMERALS

10: Input unit
20: Control unit
21: Entire laminate physical property calculation unit
22: Expansion coefficient and warpage calculation unit
30: Display
40: Storage unit

1. A system for predicting physical properties of a multilayer material having n laminated films (n is an integer of 2 or more), comprising:
an input unit configured for inputting input values including any one or more of an elastic modulus (E^{k}) of each layer (k), a Poisson's ratio (ν^{k}) of each layer (k), a shear modulus (G^{k}) of each layer (k), a thickness (Z^{k}) of each layer (k), or a lamination angle (θ^{k}) of each layer (k), coefficients of thermal expansion (α^{k}_{1,2}) or coefficients of water expansion (β^{k}_{1,2}) of each layer (k), a temperature change (ΔT), or a humidity change (ΔC);
a control unit configured to calculate the physical properties of the multilayer material by applying input values to the input unit;
a display connected to the control unit; and
a storage unit connected to the control unit,
wherein the control unit is configured to calculate any one or more of a coefficient of thermal expansion (α) of the multilayer material, a coefficient of water expansion (β) of the multilayer material, or a warpage of the multilayer material by processing values input to the input unit.

2. The system of claim 1, wherein the values input to the input unit comprise any one or more of elastic moduli (E^{k}_{1,2}) in the machine direction (1) or transverse direction (2) of each layer (k),
Poisson's ratios (ν^{k}_{1,2}) in the machine direction (1) or the transverse direction (2) of each layer (k),
shear moduli (G^{K}_{1,2}) in the machine direction (1) or the transverse direction (2) of each layer (k),
an angle (θ^{k}) in the machine direction (1) of each layer with respect to the x direction of the multilayer material, wherein the x direction means an arbitrarily set direction in a plane of the multilayer material,
a thickness (Z^{k}) of each layer (k); or
any one or more of coefficients of thermal expansion (α^{k}_{1,2}), coefficients of water expansion (β^{k}_{1,2}), a temperature change (ΔT), or a humidity change (ΔC) of each layer (k).

3. The system of claim 1, wherein the input unit is configured for inputting elastic moduli (E^{k}_{1,2}) in a machine direction (1) and a transverse direction (2) of each layer (k), Poisson's ratios (ν^{k}_{1,2}) in the machine direction (1) and the transverse direction (2) of each layer (k), shear moduli (G^{K}_{1,2}) in the machine direction (1) and the transverse direction (2) of each layer (k), an angle (θ^{k}) in the machine direction (1) of each layer with respect to the x direction of the multilayer material, wherein the x direction means an arbitrarily set direction in a plane of the multilayer material, a thickness (Z^{k}) of each layer (k), coefficients of thermal expansion (α^{k}_{1,2}), coefficients of water expansion (β^{k}_{1,2}), a temperature change (ΔT), and a humidity change (ΔC) of each layer (k).

4. The system of claim 1, wherein the control unit is configured to:
calculates stiffness matrices ([Q]^{k}_{1,2}) in the machine direction (1) and transverse direction (2) of each layer (k) using elastic moduli (E^{k}_{1,2}), Poisson's ratios (ν^{k}_{1,2}), and shear moduli (G^{k}_{1,2}),
set inverse matrices ([S]^{k}_{1,2}) for the stiffness matrices ([Q]^{k}_{1,2}) in the machine direction (1) and the transverse direction (2) of each layer (k),
reset stiffness matrices ([Q]^{k}_{x,y}) of each layer (k) by reflecting a lamination angle (θ^{k}) of the multilayer material in the stiffness matrices ([Q]^{k}_{1,2}),
calculates stiffness matrices ([A]_{x,y}, [B]_{x,y}, [D]_{x,y}) of the multilayer material using the values of the reset stiffness matrices by receiving the thickness information of each layer (k),
set compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material,
calculate free lamina hydrothermal strains (e^{k}_{1,2}) generated by water expansion of each layer (k) in a major direction of each layer (k) using coefficients of thermal expansion (α^{k}_{1,2}), coefficients of water expansion (β^{k}_{1,2}), a temperature change (ΔT), and a humidity change (ΔC) of each layer (k),
calculate hygrothermal strain transformations (e^{k}_{x,y,s}) of each layer (k) by reflecting a lamination angle (θ^{k}) of the multilayer material in the free lamina hydrothermal strains (e^{k}_{1,2}),
calculate hygrothermal forces (N^{HT}_{x,y,s}) and hygrothermal moments (M^{HT}_{x,y,s}), generated in the multilayer material, based on the hygrothermal strain transformations (e^{k}_{x,y,s}) of the multilayer material, the stiffness matrices ([Q]^{k}_{x,y}) of the multilayer material, which is the entire laminate, and the thickness (Z^{k}) of each layer (k),
form total forces (/N) and total moments (/M) by adding external forces (N,M) to the hygrothermal forces (N^{HT}_{x,y,s}) and the hygrothermal moments (M^{HT}_{x,y,s}), and
calculate a coefficient of thermal expansion (α) and coefficient of water expansion (β) of the multilayer material using the total forces (/N) and the total moments (/M), and the compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material.

5. The system of claim 4, wherein the control unit is configured to calculate strains (∈⁰_{x,y}) and curvatures (k_{x,y,s}) of a middle plane using the total forces (/N) and the total moments (/M), and the compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material, and
calculates a warpage of the multilayer material by utilizing the curvature (k_{x,y,s}) of the middle plane, and sample size (x,y) information.

6. The system of claim 1, wherein the control unit is further configured to calculate elastic moduli (E_{x,y}), shear moduli (G_{x,y}), and Poisson's ratios (ν_{x,y}) of the multilayer material using the total thickness (h) of the multilayer material and the values of compliance matrices ([a]_{x,y}, [b]_{x,y}, [c]_{x,y}, [d]_{x,y}).

7. The system of claim 1, wherein the input unit is configured for inputting elastic moduli (E^{k}_{1,2}) in a machine direction (MD, 1) and a transverse direction (TD, 2) of each layer (k), Poisson's ratios (ν^{k}_{1,2}) in the machine direction (1) and the transverse direction (2) of each layer (k), shear moduli (G^{k}_{1,2}) in the machine direction (1) and the transverse direction (2) of each layer (k), an angle (θ^{k}) in the machine direction (1) of each layer with respect to the x direction of the multilayer material, wherein the x direction means an arbitrarily set direction in a plane of the multilayer material, a thickness (Z^{k}) of each layer (k), coefficients of thermal expansion (α^{k}_{1,2}) and coefficients of water expansion (β^{k}_{1,2}) of each layer (k), a temperature change (ΔT), and a humidity change (ΔC).

8. The system of claim 1, wherein the control unit is configured to:
calculate stiffness matrices ([Q]^{k}_{1,2}) in the machine direction (1) and transverse direction (2) of each layer (k) using elastic moduli (E^{k}_{1,2}), Poisson's ratios (ν^{k}_{1,2}), and shear moduli (G^{k}_{1,2}),
set inverse matrices ([S]^{k}_{1,2}) for the stiffness matrices ([Q]^{k}_{1,2}) in the machine direction (1) and the transverse direction (2) of each layer (k),
reset stiffness matrices ([Q]^{k}_{x,y}) of each layer (k) by reflecting a lamination angle (θ^{k}) of the multilayer material in the stiffness matrices ([Q]^{k}_{1,2}),
calculate stiffness matrices ([A]_{x,y}, [B]_{x,y}, [D]_{x,y}) of the multilayer material using the values of the reset stiffness matrices by receiving the thickness information of each layer (k),
set compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material,
calculate elastic moduli (E_{x,y}), shear moduli (G_{x,y}), and Poisson's ratios (ν_{x,y}) of the multilayer material using the total thickness (h) of the multilayer material and the values of the compliance matrices ([a]_{x,y}, [b]_{x,y}, [c]_{x,y}, [d]_{x,y}),
calculate free lamina hydrothermal strains (e^{k}_{1,2}) generated by water expansion of each layer (k) in a major direction of each layer (k) using coefficients of thermal expansion (α^{k}_{1,2}), coefficients of water expansion (β^{k}_{1,2}), a temperature change (ΔT), and a humidity change (ΔC) of each layer (k),
calculate hygrothermal strain transformations (e^{k}_{x,y,s}) of each layer (k) by reflecting a lamination angle (θ^{k}) of each layer (k) in the free lamina hydrothermal strains,
calculate hygrothermal forces (N^{HT}_{x,y,s}) and hygrothermal moments (M^{HT}_{x,y,s}) generated in the multilayer material based on the hygrothermal strain transformations (e^{k}_{x,y,s}) of each layer (k), the stiffness matrices ([Q]^{k}_{x,y}) of each layer (k), and the thickness (Z^{k}) of each layer (k),
form total forces (/N) and total moments (/M) by adding external forces (N,M) to the hygrothermal forces (N^{HT}_{x,y,s}) and the hygrothermal moments (M^{HT}_{x,y,s}),
calculate a coefficient of thermal expansion (α) and coefficient of water expansion (β) of the multilayer material using the total forces (/N) and the total moments (/M), and the compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material,
calculate strains (∈⁰_{x,y}) and curvatures (k_{x,y,s}) of a middle plane using the total forces (/N) and the total moments (/M), and the compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material, and
calculate a warpage of the multilayer material by utilizing the curvature (k_{x,y,s}) of the middle plane, and sample size (x,y) information.

9. A method of predicting the physical properties of a multilayer material having n laminated films (n is an integer of 2 or more), comprising:
inputting input values including any one or more of an elastic modulus (E^{k}) of each layer (k), a Poisson's ratio (ν^{k}) of each layer (k), a shear modulus (G^{k}) of each layer (k), a thickness (Z^{k}) of each layer (k), or a lamination angle (θ^{k}) of each layer (k), coefficients of thermal expansion (α^{k}_{1,2}) or coefficients of water expansion (β^{k}_{1,2}) of each layer (k), a temperature change (ΔT), or a humidity change (ΔC); and
calculating any one or more output values of a coefficient of thermal expansion (α) of the multilayer material, a coefficient of water expansion (β) of the multilayer material, or a warpage of the multilayer material by applying the input values.

10. The method of claim 9, wherein, in the inputting of input values,
the input values comprise any one or more of elastic moduli (E^{k}_{1,2}) in the machine direction (1) or transverse direction (2) of each layer (k),
Poisson's ratios (ν^{k}_{1,2}) in the machine direction (1) or transverse direction (2) of each layer (k),
shear moduli (G^{k}_{1,2}) in the machine direction (1) or transverse direction (2) of each layer (k),
an angle (θ^{k}) in the machine direction (1) of each layer with respect to the x direction of the multilayer material, wherein the x direction means an arbitrarily set direction in a plane of the multilayer material, and
a thickness (Z^{k}) of each layer (k); and
any one or more of coefficients of thermal expansion (α^{k}_{1,2}), coefficients of water expansion (β^{k}_{1,2}), a temperature change (ΔT), or a humidity change (ΔC) of each layer (k).

11. The method of claim 9, wherein the inputting of input values comprises inputting elastic moduli (E^{k}_{1,2}) in the machine direction (1) and transverse direction (2) of each layer (k), Poisson's ratios (ν^{k}_{1,2}) in the machine direction (1) and the transverse direction (2) of each layer (k), shear moduli (G^{k}_{1,2}) in the machine direction (1) and the transverse direction (2) of each layer (k), an angle (θ^{k}) in the machine direction (1) of each layer (k) with respect to the x direction of the multilayer material, and a thickness (Z^{k}) of each layer (k).

12. The method of claim 9, wherein the calculating of output values comprises calculating stiffness matrices ([Q]^{k}_{1,2}) in the machine direction (1) and transverse direction (2) of each layer (k) using elastic moduli (E^{k}_{1,2}), Poisson's ratios (ν^{k}_{1,2}), and shear moduli (G^{k}_{1,2});
setting inverse matrices ([S]^{k}_{1,2}) for the stiffness matrices ([Q]^{k}_{1,2}) in the machine direction (1) and the transverse direction (2) of each layer (k);
resetting stiffness matrices ([Q]^{k}_{x,y}) of the multilayer material by reflecting a lamination angle (θ^{k}) of each layer (k) in the stiffness matrices ([Q]^{k}_{1,2});
calculating stiffness matrices ([A]_{x,y}, [B]_{x,y}, [D]_{x,y}) of the multilayer material using the values of the reset stiffness matrices by receiving the thickness information of each layer (k);
setting compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material;
inputting coefficients of thermal expansion (α^{k}_{1,2}), coefficients of water expansion (β^{k}_{1,2}), a temperature change (ΔT), and a humidity change (ΔC) of each layer (k);
calculating free lamina hydrothermal strains (e^{k}_{1,2}) generated by water expansion of each layer (k) in a major direction of each layer (k) using the coefficients of thermal expansion (α^{k}_{1,2}), coefficients of water expansion (β^{k}_{1,2}), temperature change (ΔT), and humidity change (ΔC) of each layer (k);
calculating hygrothermal strain transformations (e^{k}_{x,y,s}) of the multilayer material by reflecting a lamination angle (θ^{k}) of the multilayer material in the free lamina hydrothermal strains (e^{k}_{1,2});
calculating hygrothermal forces (N^{HT}_{x,y,s}) and hygrothermal moments (M^{HT}_{x,y,s}), generated in the multilayer material, based on the hygrothermal strain transformations (e^{k}_{x,y,s}) of the multilayer material, the stiffness matrices ([Q]^{k}_{x,y}) of the multilayer material, which is the entire laminate, and the thickness (Z^{k}) of each layer (k);
forming total forces (/N) and total moments (/M) by adding external forces (N,M) to the hygrothermal forces (N^{HT}_{x,y,s}) and the hygrothermal moments (M^{HT}_{x,y,s}); and
calculating a coefficient of thermal expansion (α) and coefficient of water expansion (β) of the multilayer material using the total forces (/N) and the total moments (/M), and the compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material.

13. The method of claim 12, further comprising:
calculating calculates strains (∈⁰_{x,y}) and curvatures (k_{x,y,s}) of a middle plane using the total forces (/N) and the total moments (/M), and the compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material; and
calculating a warpage of the multilayer material by utilizing the curvature (k_{x,y,s}) of the middle plane, and sample size (x,y) information.

14. The method of claim 12, further comprising:
after the setting of the compliance matrices ([a]_{x,y}, [b]_{x,y}, [c]_{x,y}, [d]_{x,y}) (S16),
calculating elastic moduli (E_{x,y}), shear moduli (G_{x,y}), and Poisson's ratios (ν_{x,y}) of the multilayer material using the total thickness (h) of the multilayer material and the values of the compliance matrices ([a]_{x,y}, [b]_{x,y}, [c]_{x,y}, [d]_{x,y}).

15. The method of claim 9, wherein the inputting of input values comprises inputting elastic moduli (E^{k}_{1,2}) in the machine direction (1) and transverse direction (2) of each layer (k), Poisson's ratios (ν^{k}_{1,2}) in the machine direction (1) and the transverse direction (2) of each layer (k), shear moduli (G^{k}_{1,2}) in the machine direction (1) and the transverse direction (2) of each layer (k), an angle (θ^{k}) in the machine direction (1) of each layer (k) with respect to the x direction of the multilayer material, and a thickness (Z^{k}) of each layer (k).

16. The method of claim 9, wherein the calculating of output values comprises calculating stiffness matrices ([Q]^{k}_{1,2}) in the machine direction (1) and transverse direction (2) of each layer (k) using elastic moduli (E^{k}_{1,2}), Poisson's ratios (ν^{k}_{1,2}), and shear moduli (G^{k}_{1,2});
setting inverse matrices ([S]^{k}_{1,2}) for the stiffness matrices ([Q]^{k}_{1,2}) in the machine direction (1) and the transverse direction (2) of each layer (k);
resetting stiffness matrices ([Q]^{k}_{x,y}) of the multilayer material by reflecting a lamination angle (θ^{k}) of each layer (k) in the stiffness matrices ([Q]^{k}_{1,2});
calculating stiffness matrices ([A]_{x,y}, [B]_{x,y}, [D]_{x,y}) of the multilayer material using the values of the reset stiffness matrix by receiving the thickness information of each layer (k);
setting compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material;
calculating elastic moduli (E_{x,y}), shear moduli (G_{x,y}), and Poisson's ratios (ν_{x,y}) of the multilayer material using the total thickness (h) of the multilayer material and the values of the compliance matrices ([a]_{x,y}, [b]_{x,y}, [c]_{x,y}, [d]_{x,y});
inputting coefficients of thermal expansion (α^{k}_{1,2}), coefficients of water expansion (β^{k}_{1,2}), a temperature change (ΔT), and a humidity change (ΔC) of each layer (k);
calculating free lamina hydrothermal strains (e^{k}_{1,2}) generated by water expansion of each layer (k) in a major direction of each layer (k) using the coefficients of thermal expansion (α^{k}_{1,2}), coefficients of water expansion (β^{k}_{1,2}), temperature change (ΔT), and humidity change (ΔC) of each layer (k) (S22);
calculating hygrothermal strain transformations (e^{k}_{x,y,s}) of the multilayer material by reflecting a lamination angle (θ^{k}) of the multilayer material in the free lamina hydrothermal strains (e^{k}_{1,2});
calculating hygrothermal forces (N^{HT}_{x,y,s}) and hygrothermal moments (M^{HT}_{x,y,s}), generated in the multilayer material, based on the hygrothermal strain transformations (e^{k}_{x,y,s}) of the multilayer material, the stiffness matrices ([Q]^{k}_{x,y}) of the multilayer material, which is the entire laminate, and the thickness (Z^{k}) of each layer (k);
forming total forces (/N) and total moments (/M) by adding external forces (N,M) to the hygrothermal forces (N^{HT}_{x,y,s}) and the hygrothermal moments (M^{HT}_{x,y,s});
calculating a coefficient of thermal expansion (α) and coefficient of water expansion (β) of the multilayer material using the total forces (/N) and the total moments (/M), and the compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material;
calculating calculates strains (∈⁰_{x,y}) and curvatures (k_{x,y,s}) of a middle plane using the total forces (/N) and the total moments (/M), and the compliance matrices ([a]_{x,y}, [b]_{x,y}, [c]_{x,y}, [d]_{x,y}) for the stiffness matrices ([A]_{x,y}, [B]_{x,y}, [D]_{x,y}) of the multilayer material; and
calculating a warpage of the multilayer material by utilizing the curvature (k_{x,y,s}) of the middle plane, and sample size (x,y) information.
